(19) United States
(12) Patent Application Publication
(10) Pub. No.: US 2024/0108381 A1
(43) Pub. Date: Apr. 4, 2024

(54) SYSTEMS AND METHODS FOR APPLYING TENSION TO THE SPINE

(71) Applicant: The Board of Regents of the University of Texas System, Austin, TX (US)

(72) Inventor: Edward P. Southern, Cuero, TX (US)

(21) Appl. No.: 18/376,796

(22) Filed: Oct. 4, 2023

Related U.S. Application Data
(60) Provisional application No. 63/413,015, filed on Oct. 4, 2022.

(51) Int. Cl. A61B 17/70

(52) U.S. Cl. CPC A61B 17/7022; A61B 17/7014; A61B 2017/00862

(57) ABSTRACT

In one embodiment, a spinal tethering system includes multiple bone anchors each configured to be secured to a vertebra of the spine, a tether configured to connect and apply tension to the bone anchors for the purpose of modulating growth of the spine, and a clutch mechanism configured to selectively release the tether to enable adjustment of the tether and, therefore, adjustment of the tension that the tether applies to the bone anchors.

### CROSS-REFERENCE TO RELATED APPLICATION

This application claims priority to co-pending U.S. Provisional Application Ser. No. 63/413,015, filed Oct. 4, 2022, which is hereby incorporated by reference herein in its entirety.

### BACKGROUND

Clinical adoption of systems designed to treat scoliosis in children using growth modulation of the spine seems inevitable in the United States for both anterior and posterior sides of the spine. Several such systems are currently being developed and marketed under a humanitarian device exemption of the Food and Drug Administration (FDA). Unfortunately, these systems can create high, cyclical loads on the bone anchor points, which can lead to failure of the bone-anchor interface as well as breaking of the tethers that extend between the bone anchors. In addition, it can be difficult to connect the tethers of such systems to the anchors, especially in anterior vertebral body tethering systems. It can, therefore, be appreciated that there is a need for a spinal tethering system that does not overload the bone-anchor interfaces or the tethers of the system, and with which the tethers can be more easily connected within the system.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a front/top perspective view of an embodiment of a spinal tethering system that, for example, can be used to treat scoliosis.

FIG. 2 is a front/bottom perspective view of the spinal tethering system of FIG. 1.

FIG. 3 is a rear/bottom perspective view of the spinal tethering system of FIG. 1.

FIG. 4 is a side/top perspective view of an embodiment of a bone anchor that can, for example, be used in the system of FIG. 1.

### DETAILED DESCRIPTION

As described above, there is a need for a spinal tethering system that does not overload the bone-anchor interfaces or the tethers of the system, and with which the tethers can be more easily connected within the system. Disclosed herein are example embodiments of such a system as well as example embodiments of methods for tensioning the spine using such a system. In some embodiments, a spinal tethering system comprises multiple bone anchors each configured to be secured to a vertebra of the spine, a tether configured to apply tension to the bone anchors for the purpose of modulating growth of the spine, and a clutch mechanism configured to selectively release the tether to enable adjustment of the tension that the tether applies to the anchors. In some embodiments, the bone anchors include rotatable pulleys around which the tether can be wrapped. In some embodiments, the clutch mechanism comprises a sheath through which the tether passes that, in its default, engaged state, prevents relative movement between the sheath and the tether and, therefore, prevents unintended changes in the tension that the tether applies, but that can be selectively disengaged by a medical professional, such as a surgeon, so as to enable desired adjustment of the tension that the tether applies.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

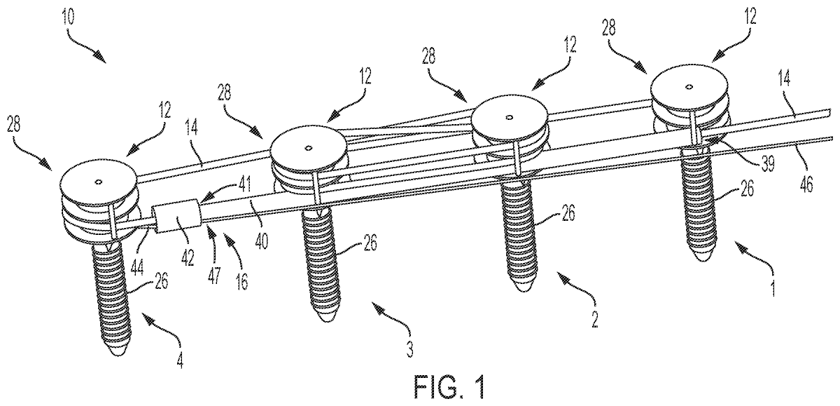

FIGS. 1-3 illustrate an example embodiment of a spinal tether system 10 that can, for example, be used to treat scoliosis by modulating growth of the spine. As shown in those figures, the system 10 generally includes multiple bone anchors 12 that are each configured to be secured to (i.e., threaded into) a vertebra of the spine, a tether 14 configured to apply tension to the bone anchors (and, therefore, the vertebrae to which they are secured), and a clutch mechanism 16 configured to selectively release the tether to enable adjustment of the tether and, therefore, the tension that it applies to the anchors. In the example of FIGS. 1-3, the system 10 includes four bone anchors 12, which have also been numbered 1, 2, 3, and 4 to enable independent identification of each anchor to facilitate description of the system. It is noted that, although the system 10 is shown and described herein as comprising four anchors 12, a greater or lesser number of anchors can be used depending upon what is needed for the particular application.

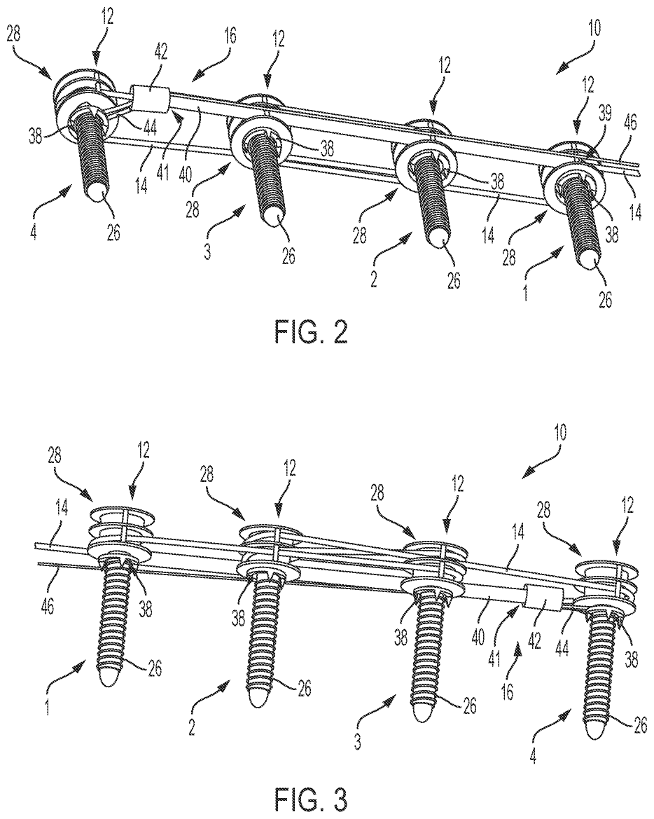

The first bone anchor 1 can be considered to be the most proximal anchor, while the fourth bone anchor 4 can be considered to be the most distal anchor. As will become apparent from the discussion that follows, those designations are used as they characterize the anchors 12 in terms their proximity to a medical professional who sets or adjusts the tension applied to the anchors by the tether 14. Accordingly, in the context of the present disclosure, the left end of the system 10 as depicted in FIG. 1 can be considered to be the distal end of the system, and the right end of the system as depicted in FIG. 1 can be considered to be the proximal end of the system. It is noted that the terms “distal” and “proximal” are occasionally to describe particular sides or ends of other components of the system 10 in accordance with the above convention. Furthermore, to facilitate description of the system 10, FIGS. 1 and 2 can be considered to show a front side of the system, while FIG. 3 can be considered to show a rear side of the system.

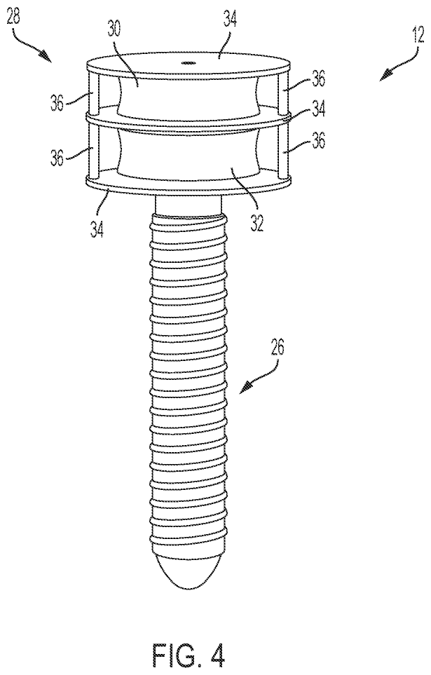

FIG. 4 illustrates an example embodiment for the bone anchors 12. As shown in that figure, the bone anchor 12 is formed as a bone screw having a threaded shaft 26 that extends from a head 28 of the anchor. The threaded shaft 26 is configured to be screwed into the vertebral body of a patient's spine and can either be solid or hollow. In embodiments in which the shafts 26 are solid, the shafts can, for example, have diameters of approximately 6 to 8 mm. In embodiments in which the shafts 26 are hollow, the shafts can, for example, have diameters of approximately 8 to 12 mm. Generally speaking, larger diameter shafts 26 provide greater lateral migration resistance, which is of primary importance once the tether 14 has been tensioned.

In some embodiments, the shafts 26 can have a polished finish. In other embodiments, the shafts 26 can have a plasma sprayed hydroxyapatite surface finish or another surface treatment, such as ion-bombardment, to improve the osseointegration and biomechanics of the anchor by facilitating bonding to the shafts at the cellular level. In further embodiments, the shafts 26 can have porous channels that enable bone to grow into the shaft to increase axial torsional and transverse load bearing.

In still further embodiments, the threads of the shafts 26 can have square shaped cross-section with filleted corners. By way of example, the threads can have a pitch of approximately 0.8 to 1.6 mm, a depth of approximately 0.44 to 0.5 mm, and a width or thickness of approximately 0.18 to 0.50 mm.

The head 28 of the anchor 12 in the example of FIG. 4 includes two horizontally oriented and vertically stacked (in the context of FIG. 4) freely rotatable pulleys, including a first or upper pulley 30 and a second or lower pulley 32. Although the anchor 12 is illustrated as comprising two pulleys 30, 32, it is noted that one or more of the anchors 12 of the system 10 can include a greater or lesser number of pulleys, again depending upon what is needed for the particular application.

Each of the pulleys 30, 32 can independently rotate relative to the shaft 26 of the anchor 12 about a vertical axle (not visible in the figure) that extends upwardly from the shaft along a central longitudinal axis of the shaft, which is concentric with the central longitudinal axis of the anchor. In some embodiments, the pulley axles can comprise bearingless sheave axles having low-wear polymer bushings. In some embodiments, each pulley 30, 32 can have an outer diameter that is at least approximately five times larger than the diameter of the tether 14 and, in some cases, eight to twelve times larger than the diameter of the tether. As for the U-shaped grooves of the pulleys 30, 32 about which the tether 14 wraps, the base of the groove can have a diameter that is approximately 1.1 to 1.2 times larger than the diameter of the tether. the intended tether diameter, and the groove can have a depth that is approximately 0.32 to 0.5 times the diameter of the tether.

As described below, the tether 14 can be wrapped around one or both of the pulleys 30, 32 (or a greater number of pulleys if the anchor 12 is so equipped) for the purpose of applying tension to the anchors and, therefore, the vertebrae to which they are secured. Also shown in FIG. 4 are horizontally oriented flanges 34 configured as thin circular discs that are positioned above and below each pulley 30, 32. While the pulleys 30, 32 can freely rotate relative to the shaft 26, the flanges 34 are fixed and, therefore, cannot rotate. Extending between the flanges 34 are vertically oriented retainer rods 36 that can be used to retain the tether 14 in place relative to a pulley 30, 32 of the anchor 12. When provided, the rods 36 can extend from one plate 34 to the next at the outer peripheral edges of the flanges.

Referring next to FIGS. 2 and 3, bone staples 38 can be provided around a neck of the shaft 26 of each bone anchor 12 where the shaft extends from the head 28 of the anchor. When present, the staples 38 embed into the bone of the vertebrae to reduce the likelihood of its associated anchor 12 loosening as a result of shear forces applied to the vertebral bodies by the anchor.

With reference next to FIGS. 1-3, the tether 14 can be made of a high tensile strength elastic or inelastic material. By way of example, the tether 14 can comprise one or more fibers, strands, or yarns composed at least in part of an elastic polymer material, such as a polyethylene terephthalate (PET) or a polyamide. Another example polymer material is aramid materials, such as a meta-aramid and/or a para-aramid materials. In other embodiments, the tether 14 can comprise a natural material, such as silk. Generally speaking, the tether 14 is fixedly attached to a pulley of the first bone anchor 1, wraps around each of the bone anchors 12 at least once, and then passes through the clutch mechanism 16 such that a length of tether extends from the mechanism. Notably, the free end of the tether 14 can be left inside of the patient at an easily accessible location, such as just below the patient's skin, to enable a medical professional to easily access and adjust the tether at some point after the system 10 was initially implanted. Accordingly, if adjustments in tension are required at a later date as the patient grows, such adjustments can be made with minimal invasiveness.

With continued reference to FIGS. 1-3, the clutch mechanism 16 of the illustrated embodiment includes a braided sheath 40 through which the tether 14 extends. As shown most clearly in FIGS. 1 and 2, the sheath 40 can have a length that is shorter than the distance between the first bone anchor 1 and the fourth bone anchor 4 of the system 10 and can extend between the pulleys and retainer rods of multiple bone anchors. In the illustrated example, the sheath 40 extends from a point between the third bone anchor 3 and the fourth bone anchor 4, and passes between the lower pulleys 32 of the first, second, and third bone anchors 1, 2, and 3.

As is most clearly apparent from FIG. 2, a proximal end 39 of the sheath 40 is fixedly attached to the first bone anchor 1 (e.g., to one of its retainer rods 36), and a distal end 41 of the sheath terminates in an end cap 42 to which an elastic tensioning cord 44 and an inelastic release cord 46 are attached. As shown in FIG. 2, the tensioning cord 44 is looped around the shaft 26 of the fourth bone anchor 4 near that anchor's bone staple 38 and each end of the cord is fixedly attached to the end cap 42. As for the release cord 46, its distal end 47 is fixedly attached to the cap 42 and the cord extends away from the cap along the same or a similar direction as the tether 14. As with the tether 14, the proximal free end of the release cord 46 can be positioned in an easily accessible location within the patient, such as the same location as the free end of the tether, so that the cord can be used to selectively disengage the clutch mechanism 16 when a medical professional wishes to adjust the tension in the tether.

By default, the clutch mechanism 16 is in an engaged state in which the tether 14 is locked in place and cannot move relative to (i.e., pass through) the braided sheath 40 of the mechanism to ensure that, once the desired tension has been applied to the bone anchors 12 with the tether, the tether cannot loosen. The clutch mechanism 16 prevents such relative movement because of the friction that exists between the sheath 40 and the tether 14 when the clutch mechanism 16 is engaged. In that state, the sheath 40 is in an extended or stretched orientation that results from its attachment to the first bone anchor 1 and the tension applied to the end cap 42 and, therefore, the sheath by the tensioning cord 44. When the sheath 40 is stretched in that manner, the diameter of the sheath is relatively small and the sheath tightly grips the tether 14 so that it is trapped in similar manner to the way in which one's fingers are trapped by what is commonly referred to as a “Chinese finger trap” when one tries to pull their fingers apart while they are placed inside the sheath of the trap.

When the end cap 32 is pulled in the proximal direction away from the fourth bone anchor 4 (to the right in the orientation of FIG. 1) against the tension applied by the tensioning cord 44, however, the sheath 40 shortens and its diameter increases so as to reduce or eliminate the friction between the sheath and the tether 14, effectively releasing the tether so that it is unlocked and can freely move relative to (i.e., through) the sheath. Therefore, a medical professional can adjust the tension applied to the bone anchors 12 by first pulling on the release cord 46 to disengage the clutch mechanism 16 and, while maintaining tension on the release cord, either pull the tether 14 in the proximal direction away from the fourth bone anchor 4 to increase the tension applied to the bone anchors 12 or enable the tether to be pulled in the distal direction toward the fourth bone anchor to enable a greater length of the tether to be wrapped around the anchors and decrease the tension it applies to those anchors. Once the desired amount of tension has been achieved, the medical professional can gradually release the release cord 46 while holding the tether 14 in place to enable the sheath 40 to stretch under the pulling force of the tensioning cord 44, decrease in diameter, and trap the tether 14 in its new position. Notably, in cases in which the tension is to be adjusted at a time after the system 10 has been implanted and the proximal free ends of the tether 14 and the release cord 46 are embedded within the patient, the free ends can be can be accessed by a medical professional by making a small incision through the skin at the point at which the free ends lie.

As noted above, the tether 14 wraps around the head 28 of each of the bone anchor 12 of the implanted system 10 at least once. In some embodiments, such as that illustrated in FIGS. 1-3, the tether 14 wraps around the head 28 of one or more of the bone anchors 12 multiple times, for example, wrapping around multiple pulleys of the anchors. The example wrapping scheme illustrated in FIGS. 1-3 will next be described in detail.

As best depicted in FIG. 3, which shows what has been designated as the rear side of the system 10, the distal end of the tether 14 is fixedly attached to the lower pulley 32 of the first bone anchor 1 (refer to FIG. 4 for references to the upper and lower pulleys 30, 32). The tether 14 wraps around a portion of that pulley 32 and then extends to and along the lower pulley 32 of the second bone anchor 2, and then onto the lower pulley 32 of third bone anchor 3 about which the tether wraps. Notably, in embodiments such as that illustrated in FIGS. 1-3 in which the heads 28 of the bone anchors 12 include retainer rods 36, the tether 14 passes between the retainer rods and their associated pulleys. Accordingly, in accordance with the above description, the tether 14 travels between the retainer rods 36 and the lower pulleys 32 of the first, second, and third anchors 1, 2, and 3, in that order.

Referring next to FIG. 1, which shows what has been designated as the front side of the system 10, after wrapping around the lower pulley 32 of the third bone anchor 3, the tether 14 extends to and wraps around the lower pulley 32 of the second bone anchor 2. With reference again to FIG. 3, the tether 14 then extends upward from the lower pulley 32 of the second bone anchor 2 and wraps around the upper pulley 30 of the third bone anchor 3. From that pulley 30, the tether 14 next extends to and wraps around the upper pulley 30 of the second bone anchor 2, as shown in FIG. 1. Referring again to FIG. 3, the tether next extends from the upper pulley 30 of the second bone anchor 2, to and along the upper pulley 30 of the third bone anchor 3, and down to the lower pulley 32 of the fourth bone anchor 4.

Referring again to FIG. 1, the tether 14 then wraps around the distal portion of the lower pulley 32 of the fourth bone anchor 4 and then extends through the braided sheath 40 of the clutch mechanism 16. The tether 14 passes through the sheath 40 and extends out from the proximal end 39 of the sheath with enough length to enable a medical professional to grip the free end of the tether for the purpose of setting or adjusting the tension applied by the tether to the bone anchors 12.

From the above description, it can be appreciated that, while the tether 14 wraps around a single pulley of the first bone anchor 1 and a single pulley of the fourth bone anchor 4 and, therefore, only wraps around the heads 28 of those anchors once, the tether wraps around both pulleys of the second bone anchor 2 and the third bone anchor 3 and, therefore, wraps around the heads 28 of those anchors twice. When the tether 14 wraps around the head 28 of a bone anchor 12 multiple times as it does with the second and third bone anchors 3 and 4 in the example of FIGS. 1-3, a mechanical advantage is obtained that can be used to apply different tensile forces to different bone anchors. Accordingly, the tensile forces applied to the bone anchors 12 can be custom tailored for the particular application and the needs of a particular patient by carefully choosing the manner and number of times the tether 14 wraps around each anchor.

While a specific embodiment and configuration of a spinal tethering system has been described in detail above, it is noted that that system is merely an example. Many alternative embodiments and configurations can be created based upon the principles described herein.

1. A spinal tethering system comprising:
multiple bone anchors each configured to be secured to a vertebra of the spine;
a tether configured to connect and apply tension to the bone anchors for the purpose of modulating growth of the spine; and
a clutch mechanism configured to selectively release the tether to enable adjustment of the tether and, therefore, adjustment of the tension that the tether applies to the bone anchors.

2. The system of claim 1, wherein at least one of the bone anchors comprises a freely rotatable pulley around which the tether can be wrapped.

3. The system of claim 2, wherein the freely rotatable pulley is configured to rotate about a central longitudinal axis of the at least one anchor.

4. The system of claim 1, wherein at least one of the bone anchors comprises multiple freely rotatable pulleys around which the tether can be wrapped.

5. The system of claim 1, wherein the freely rotatable pulleys are configured to rotate about a central longitudinal axis of the at least one anchor.

6. The system of claim 5, wherein the freely rotatable pulleys are configured to rotate independently of each other.

7. The system of claim 5, wherein the freely rotatable pulleys are arranged in a vertically stacked configuration.

8. The system of claim 1, wherein the tether comprises a polymer material.

9. The system of claim 8, wherein the polymer material is an elastic polymer material.

10. The system of claim 1, wherein the tether is the only tether used in the system and the only element that applies tension to the bone anchors.

11. The system of claim 1, wherein the clutch mechanism comprises a braided sheath through which the tether passes.

12. The system of claim 11, wherein the braided sheath is fixedly attached to a proximal bone anchor of the system and wherein the clutch mechanism further comprises a tensioning cord connected to a distal end of the braided sheath and to a distal bone anchor of the system that stretches the sheath to reduce a diameter of the sheath to a point at which the sheath traps the tether in place.

13. The system of claim 12, wherein the clutch mechanism further comprises a release cord connected to the distal end of the braided sheath and configured to shorten the sheath and increase its diameter to a point at which the tether can freely pass through the sheath when the release cord is pulled.

14. A bone anchor comprising:
a threaded shaft configured to be threaded into bone; and
a head provided at an end of the threaded shaft, the head comprising a freely rotatable pulley around which a tether can be wrapped.

15. The bone anchor of claim 14, wherein the freely rotatable pulley is configured to rotate about a central longitudinal axis of the anchor.

16. The bone anchor of claim 14, wherein anchor comprises multiple freely rotatable pulleys around which a tether can be wrapped.

17. The bone anchor of claim 16, wherein the freely rotatable pulleys are configured to rotate independently of each other about a central longitudinal axis of the at least one anchor.

18. The bone anchor of claim 17, wherein the freely rotatable pulleys are arranged in a vertically stacked configuration.

19. A method for applying tension to the spine, the method comprising:
implanting multiple bone anchors into vertebrae of the spine, each bone anchor including at least one freely rotatable pulley configured to rotate about a central longitudinal axis of the anchor;
wrapping a tether around the freely rotatable pulleys of the bone anchors in a predetermined pattern configured to apply a desired degree of tension to each bone anchor; and
securing the tether in place once a desired degree of tension has been applied with the tether using a clutch mechanism.

20. The method of claim 19, further comprising later adjusting the degree of tension applied to the bone anchors by disengaging the clutch mechanism, adjusting the length of the tether that is wrapped around the freely rotatable pulleys to change the degree of tension applied to the bone anchors, and then re-engaging the clutch mechanism to secure the tether in place.
